(19) United States
(12) Patent Application Publication
(10) Pub. No.: US 2022/0325317 A1
(43) Pub. Date: Oct. 13, 2022

(54) METHODS FOR GENERATING A POPULATION OF POLYNUCLEOTIDE MOLECULES

(71) Applicant: QUEEN MARY UNIVERSITY OF LONDON, London (GB)

(72) Inventors: Gabriella Ficz, London (GB); Emily Saunderson, London (GB)

(73) Assignee: QUEEN MARY UNIVERSITY OF LONDON, London (GB)

(21) Appl. No.: 17/634,444

(22) PCT Filed: Aug. 12, 2020
(86) PCT No.: PCT/GB2020/051917

§ 371 Date: Feb. 10, 2022

(30) Foreign Application Priority Data
Aug. 12, 2019 (GB) 1911515.3

(51) Int. Cl. C12Q 1/6806; C12Q 1/6874

(52) U.S. Cl. CPC C12Q 1/6806; C12Q 1/6874

(57) ABSTRACT

The present invention relates to novel methods for generating a population of double-stranded polynucleotide molecules from a sample containing at least one polynucleotide.

### FIELD OF THE INVENTION

The present invention relates to novel methods for generating a population of double-stranded polynucleotide molecules from a sample containing at least one polynucleotide.

### BACKGROUND OF THE INVENTION

Whole genome sequencing (WGS) has radically changed medical diagnostics and research and is a rapidly evolving technology platform. Illumina sequencing technologies facilitated expanding investigations from a single-region, single-gene approach to interrogating the whole genome simultaneously. While this approach is cost effective, WGS of fragmented genomic DNA is associated with sequencing and mapping artefacts, which are significantly more prevalent in formalin-fixed paraffin-embedded (FFPE) material. FFPE treatment is routinely used to preserve clinical specimens, as well as archaeological or historic samples. However it can result in extensive DNA damage (particularly DNA crosslinks and deamination of cytosines) and fragmentation, leading to poor quality sequencing data which renders many samples unusable for WGS. Consequently, large sequencing efforts such as ‘The 100,000 Genomes Project’ led by Genomics England have proposed that collection of fresh tissue should be standard of care in modern cancer diagnostics. Nevertheless, for retrospective studies FFPE tissues are often the only material available, therefore there remains a need to develop new methodology that can improve sequencing quality.

There are numerous WGS library preparation methods available to researchers, and these differ in their price, preparation time and recommended input material. Most library preparation methods for WGS rely on attaching short double stranded DNA (dsDNA) oligos to fragmented genomic dsDNA isolated from a fresh or FFPE sample of choice. The gold standard methods for WGS library preparation sold by major biotech companies continue to be improved over time in order to be applicable for very low amounts of input DNA, provided this material is of good quality (such as that isolated from fresh tissues or cells). One limitation of these kits is that the adaptor ligation step is inefficient and will not recover single stranded DNA (ssDNA).

An increasingly important extension to the WGS work-flow in academic research is a follow up method called targeted sequencing. This is used to look in greater depth (i.e. tens to thousands of reads per DNA base) at specific areas of the genome with mutations-of-interest identified from WGS (which gives tens to thousands of reads per DNA base). This is important as mutations do not always have 100% penetrance (i.e. they may not be found in all cells, particularly for disease-relevant mutations); in fact, many functionally relevant mutations are at a low frequency (i.e. less than 50%), which WGS can miss due to limited coverage per DNA base. Increasingly, patient biopsies are assessed using targeted sequencing to complement other well-established diagnostic techniques as there is rapidly growing clinical knowledge relating specific gene mutations (i.e. exon mutations) to patient prognosis and/or responses to treatment. Targeted sequencing of patient samples can identify the presence or absence of disease-relevant mutational hot-spots with high accuracy and low cost compared to WGS. For example, a gene panel for targeted sequencing consisting of up to 130 genes is approximately 0.015% of the human genome, therefore enabling much more data (more reads per DNA base) to be produced at a fraction of the cost of WGS.

Current methods for targeted sequencing invariably use ligation to attach oligonucleotides to sample DNA, (i.e. short dsDNA oligonucleotides). To capture the target-of-interest, standard methods often require specialised ‘chips’ which have target-of-interest oligonucleotides attached, followed by a long hybridisation step to anneal the sample DNA to the chip, which is an expensive and time consuming process. Similar to WGS sample preparation, a limitation of this approach is the loss of ssDNA.

### SUMMARY OF THE INVENTION

The invention provides a method for generating a population of double-stranded polynucleotide molecules from a sample containing at least one polynucleotide, which method does not comprise bisulfite treatment of said polynucleotide, and which method comprises:
a. Denaturing said polynucleotide to produce single stranded polynucleotide;
b. Incubating the single stranded polynucleotide from step a. with a first single-stranded oligonucleotide comprising a sequencing adaptor sequence and a primer sequence under conditions suitable for annealing of the first single-stranded oligonucleotide to the single stranded polynucleotide of step a., and then extending the primer with a polymerase to produce double-stranded polynucleotide;
c. Denaturing the double-stranded polynucleotide of step b. to produce single stranded polynucleotide;
d. Incubating the single stranded polynucleotide from step c. with a second single-stranded oligonucleotide comprising a sequencing adaptor sequence and a primer sequence under conditions suitable for annealing of the second single-stranded oligonucleotide to the single stranded polynucleotide of step c., and then extending the primer with a polymerase to produce a population of double-stranded polynucleotide molecules.

### BRIEF DESCRIPTION OF THE FIGURES

FIG. 1 shows a schematic that depicts an exemplary embodiment (Damaged DNA Adaptor Sequencing or DDAT) of the present invention whereby a DNA sequencing library is generated from a damaged DNA sample, as compared to known methods in the art for preparing DNA sequencing libraries from a damaged DNA sample. The embodiment of the present invention depicted in the right panel of FIG. 1 firstly shows the addition of enzymes SMUG1 (single-strand-selective monofunctional uracil-DNA Glycosylase) and Fpg (formamidopyrimidine [fapy]-DNA glycosylase) to the input DNA (portions A and B of FIG. 1) which remove damaged bases such as deoxyuracil and 8-oxoguanine, caused by the FFPE treatment. A short denaturation step (portion B of FIG. 1) is followed by the first strand synthesis; during this step the genomic DNA, primers and Klenow polymerase (with exonuclease activity) are gradually heated from 4° C. to 37° C. with a slow ramping speed of 4° C. per minute, before incubation at 37° C. for a further 1.5 hours (portion C of FIG. 1). The primers contain 9 random nucleotides from the 3′-end, in addition to the standard Illumina adaptor sequence, and will anneal to complementary DNA sequences present in the DNA sample. After the first strand synthesis, any remaining primers or short ssDNA fragments are digested with exonuclease I and the dsDNA is purified with AmpureXP beads. Next, the dsDNA is denatured to carry out the second strand synthesis using a second adaptor primer also containing 9 random nucleotides, with the same conditions as the first synthesis, followed by bead purification (portion C of FIG. 1). Finally, 10 PCR cycles are carried out using standard Illumina p5 and p7 indexed primers (portion D of FIG. 1). The library is purified and assessed using standard quality control methods.

FIG. 2A shows the percentage of the genome covered by sequencing reads derived from an exemplary embodiment (DDAT) of the present invention whereby a DNA sequencing library is generated from a damaged DNA sample, as compared to a known method in the art for preparing DNA sequencing libraries from a damaged DNA sample. The DDAT method resulted in a 2.5-fold increase in coverage in terms of number of reads per base in the genome.

FIG. 2B shows the distribution of insert size in sequencing reads derived from an exemplary embodiment (DDAT) of the present invention whereby a DNA sequencing library is generated from a damaged DNA sample, as compared to a known method in the art for preparing DNA sequencing libraries from a damaged DNA sample. The DDAT method resulted in a 2.5-fold increase in coverage in terms of number of reads per base in the genome. In this context, “insert” refers to the sequence of nucleotides between the paired-end adaptor sequences a DNA molecule within a sequencing library. The larger insert size generated by the exemplary DDAT method is indicative of methods of the invention capturing more of the input DNA in the sample than a standard method known previously in the art.

FIG. 2C shows sequencing reads on the Integrative Genomics Viewer. Sequencing data that has been derived according to an exemplary embodiment (DDAT) of the present invention (upper panel) shows a C>A transition (A base shown in between the dashed lines; chr 5:112838399; GRCh38; total reads=19, altered reads=9, variant allele frequency (VAF)=0.474) resulting in a stop codon in the APC gene (p.Y935*, c.2805C>A; COSMIC19031). When using the standard library preparation method (lower panel), this region is not covered by enough reads to be identified (total reads=2, altered reads=2, VAF=1).

FIG. 3 shows a bar chart that indicates sequencing library yields derived from good, poor or very poor samples when implementing an exemplary embodiment (DDAT) of the present invention as compared to known methods in the art for preparing DNA sequencing libraries from a damaged DNA sample. Greater yields of DNA can be achieved by using methods of the invention as opposed to standard methods of sequencing library preparation.

FIG. 4 shows that for all sample qualities assayed, a greater genome coverage and reads per base can be achieved by implementing an exemplary embodiment (DDAT) of the present invention as compared to a known method in the art for preparing DNA sequencing libraries from a damaged DNA sample.

FIG. 5A shows that C>T/A>G mutation ratios determined by sequencing of DNA sequencing libraries derived from good, poor, or very poor samples, are equivalent in methods of the invention that feature a base excision repair enzyme relative to a standard method known in the art for preparing DNA sequencing libraries. An exemplary embodiment of the present invention that lacked the use of a base excision repair enzyme showed an increased C>T/A>G mutation ratio relative to a standard method known in the art for preparing DNA sequencing libraries, thus indicating the use of a base excision repair enzyme in the methods of the present invention can decrease sequencing artefacts that result from damaged input DNA.

FIG. 5B shows a bar chart representing an average C>T/A>G mutation ratio across sequencing of DNA sequencing libraries derived from good, poor, or very poor samples, when assayed by a standard method known in the art for preparing DNA sequencing libraries, or methods according to the present invention with or without the use of a base excision repair enzyme.

FIG. 6 shows multiplex PCR products of DNA derived from FFPE samples run on an agarose gel to show sample quality assessed by PCR amplification of 100 bp, 200 bp, 300 bp and 400 bp fragments of the GAPDH gene. Samples shown are those used to generate sequencing libraries in the Examples of the present application with either the standard or DDAT method.

FIG. 7 shows DNA fragments size distribution within sequencing libraries prepared by a standard library preparation method (top) or DDAT (bottom) using DNA derived from FFPE samples as measured by Tapestation (Agilent) quantification.

FIG. 8 shows a bar chart that indicates median insert sizes in sequencing libraries derived from good, poor or very poor samples when implementing an exemplary embodiment (DDAT) of the present invention, with or without the addition of use of SMUG1/Fpg base excision repair enzymes, as compared to known standard methods in the art for preparing DNA sequencing libraries from a damaged DNA sample. Greater insert sizes are observed within sequencing libraries when DDAT is used as compared to the standard methods in the art. Further increases in insert sizes are observed for poor quality samples when SMUG1/Fpg base excision repair enzymes are used in accordance with the methods of the invention.

FIG. 9 shows the mean genomic coverage (average reads per base) achieved by sequencing libraries derived from good, poor or very poor samples when implementing an exemplary embodiment (DDAT) of the present invention, with or without the addition of use of SMUG1/Fpg base excision repair enzymes, as compared to known standard methods in the art for preparing DNA sequencing libraries from a damaged DNA sample. Further increases in genomic coverage are observed for poor quality samples when SMUG1/Fpg base excision repair enzymes are used in accordance with the methods of the invention.

FIG. 10 shows a bar chart depicting the effect of slow ramping rate (rate of increase in temperature from 4° C. up to the optimal temperature of the DNA-directed DNA polymerase) in the first and second extension steps on library yields (measured in terms of library molarity nM) of the method of the inventions when applied to an exemplary embodiment of the present invention (DDAT) as compared to known standard methods for preparing DNA sequencing libraries. Fast ramping rate=132° C./min; slow ramping rate=4° C./min.

FIG. 11 shows primers containing the TET2-specific sequence and the truncated P7 part of the Illumina adapter are used in the 1^{st} strand synthesis. The random N×9 bp attached to the truncated P5 part of the Illumina adapter is used in the 2^{nd} strand synthesis. The 2^{nd} strand synthesis primer will bind randomly to the new DNA strands that were generated during the 1^{st} strand synthesis. After the PCR amplification the final library will contain complete sequencing fragments, and the sequencing will commence from the P5 end, meaning that the first read will always start from a random sequence of the TET2 gene, rather than the TET2-specific primer, which is at the P7 end.

FIG. 12 shows data derived from exemplary embodiments of the invention (TDAT and DDAT) that is visualized using IGV (integrative genome viewer). The grey peaks show a summary of the sequencing reads at the TET2 gene.

FIG. 13 shows sanger sequencing data derived from an exemplary embodiment of the invention (TDAT) sequencing trace validating G/A mutation in KG-1 cells. Overlapping G and A traces show heterozygous mutation identified using the embodiment of the invention.

FIG. 14 shows data derived from an exemplary embodiment of the invention (TDAT) that is visualized using IGV (integrative genome viewer). Horizontal grey bars are indicative of reads that span the IGV visualization region. The wild type human genomic sequence can be viewed along the x axis. The TDAT method successfully identifies a G/A mutation.

### BRIEF DESCRIPTION OF THE SEQUENCES

SEQ ID NO: 1 is an exemplary first single-stranded oligonucleotide comprising a sequencing adaptor sequence and a random primer sequence (as represented by ‘N’) for annealing to a first single-stranded polynucleotide and thus enabling extension with a DNA polymerase to produce a first double-stranded polynucleotide.

SEQ ID NO: 2 is an exemplary second single-stranded oligonucleotide comprising a sequencing adaptor sequence and a random primer sequence (as represented by ‘N’) for annealing to a second single-stranded polynucleotide and thus enabling extension with a DNA polymerase to produce a second double-stranded polynucleotide.

SEQ ID NO: 3 is a sequencing library PCR primer containing a nucleotide sequence suitable for annealing to oligonucleotides coating a sequencing flow cell (e.g. Illumina® next generation sequencing technologies).

SEQ ID NO: 4 is an indexed sequencing library PCR primer containing a nucleotide sequence suitable for annealing to oligonucleotides coating a sequencing flow cell (e.g. Illumina® next generation sequencing technologies), wherein the index enables the user to pool/multiplex libraries for sequencing then subsequently bioinformatically segregate and analyse the sequencing data for each distinctly indexed library.

SEQ ID NO: 5 is an exemplary first single-stranded oligonucleotide comprising a sequencing adaptor sequence and a primer sequence for annealing a region of interest in the TET2 gene thus enabling extension with a DNA polymerase to produce a first double-stranded polynucleotide.

SEQ ID NO: 6 is an exemplary second single-stranded oligonucleotide comprising a sequencing adaptor sequence and a random primer sequence (as represented by ‘N’), preferably used when the first single-single stranded oligonucleotide is designed to anneal to a specific region of interest, for annealing to a second single-stranded polynucleotide and thus enabling extension with a DNA polymerase to produce a second double-stranded polynucleotide.

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a method” includes “methods”, and the like.

All publications, patents and patent applications cited herein, whether supra or infra, are hereby incorporated by reference in their entirety.

The present inventors have devised a method for generating a population of double-stranded polynucleotide molecules from a sample containing at least one polynucleotide.

A “population”, is used herein to refer to a plurality of molecules. “polynucleotide molecules” used herein may refer to DNA, sequences of deoxyribonucleotides, polynucleotides, polynucleotide analogs, sequences of synthetic deoxyribonucleotides, or fragments of DNA. The population of polynucleotide molecules may comprise single-stranded polynucleotide or double-stranded polynucleotide. The population of polynucleotide molecules may be cDNA. The population of polynucleotide molecules may be a DNA sequencing library. The DNA sequencing library may also comprise any one, or both, of sequencing adaptors and primers. In any of the methods described herein, the population may refer to a plurality of RNA molecules.

The method of the invention may also be used for generating a population of double-stranded polynucleotide molecules from a sample containing RNA. “RNA molecules” used herein may refer to sequences of ribonucleotides, polynucleotides, polyribonucleotides, polyribonucleotide analoges, sequences of synthetic ribonucleotides, or fragments of RNA. The RNA molecules may comprise single-stranded RNA or double-stranded RNA. The RNA molecules may by an RNA sequencing library.

A “sample” is used herein to refer to any material containing at least one polynucleotide. At least one polynucleotide may be RNA or DNA. An exemplary sample may be a soil sample, or a sample of any material or tissue obtained from a plant or animal. Preferred animal materials include hair follicles and body fluids such as blood, saliva, semen, vaginal fluids, mucus, urine or any other humoral material. The sample may be a cellular lysate. The sample may be fixed, for example by heat, immersion or perfusion. In particular, the sample may be derived from organisms, tissues, or tissue cross-sections that have been subjected to chemical fixation. For example, the sample may be of formalin-, paraformaldehyde-, osmium tetroxide-, glutaraldehyde-, alcohol-, HOPE (hepes-glutamic acid buffer-mediated organic solvent protection effect)-, or bouin solution-fixed material. The sample may be of formalin-fixed and paraffin embedded (FFPE) material. “Sample” may also refer to ‘input polynucleotide’, i.e. polynucleotide, that may have been derived from a source material that contains polynucleotide, that is to be inputted directly to the first denaturing step of the methods described herein.

The sample may contain any quantity or quality of polynucleotide. In particular, the sample may contain any quantity or quality of DNA or RNA. The sample may contain a low quantity and/or low quality of DNA or RNA. The sample may contain less than around 10 μg, less than around 5 μg, less than around 1 μg, less than around 500 ng, less than around 200 ng, less than around 100 ng, less than around 50 ng, less than around 10 ng, less than around 5 ng or less than around 1 ng of DNA or RNA. Although preferably, the sample contains between around 0.1 ng to around 100 ng, around 0.5 ng to around 20 ng, around 2 ng to around 10 ng of DNA. The sample may contain less than around 1 μg, preferably less than around 200 ng, most preferably between around 2 ng to around 10 ng of DNA or RNA. A significant proportion of the DNA or RNA may be fragmented, damaged and/or in single-stranded form.

The quality of the polynucleotide used in the method herein may be determined through the use of any known method in the art. For example, samples containing DNA could be run on an agarose gel and thus enabling the DNA contained within a sample to be visualised via the use of any appropriate method or instrument in order to determine the quality of the DNA in the sample. Visualisation may be conducted with or without prior amplification of the DNA in the sample. Samples containing DNA could be visualised and/or detected with, for example, a NanoDrop (Thermo Fisher Scientific), a TapeStation (Agilent) or Bioanalyzer (Agilent) in order to determine the quality of the DNA in the sample. DNA quality may be estimated using a multiplex PCR-based assay as well known in the art. Following a multiplex PCR-based assay, visualisation and/or detection of the DNA can be conducted by any known method or instrument in the art, for example, by applying the DNA sample to agarose gel electrophoresis, or applying the DNA sample to a NanoDrop (Thermo Fisher Scientific), a TapeStation (Agilent) or Bioanalyzer (Agilent). A low quality DNA sample, with or without prior amplification, that is optionally a multiplexed PCR-based assay, would not have detectable and/or visible PCR products when the DNA sample is assayed by any suitable method known in the art. A skilled user would understand the output data of these exemplary DNA quality assessment instruments and would be able to determine the quality of the DNA in the sample, and in particular, whether a significant proportion of the DNA in the sample is fragmented, damaged and/or in single stranded form.

Polynucleotide contained within the sample to be used in accordance with the present invention may be fragmented, damaged and/or in single-stranded form. A significant proportion of the polynucleotide in the sample may be fragmented, damaged and/or in single stranded form.

In any of the methods described herein, the sample for utilisation according to the method may contain low quantity polynucleotide and/or low quality polynucleotide, optionally wherein the sample contains less than around 1 μg, preferably less than around 200 ng, most preferably between around 2 ng to around 10 ng of polynucleotide, and/or wherein a significant proportion of the polynucleotide is fragmented, damaged and/or in single-stranded form. Said polynucleotide may be RNA or DNA.

The methods described herein may further comprise:
a. Denaturing the polynucleotide from the sample to produce single-stranded polynucleotide;
b. Incubating the single stranded polynucleotide from step a. with a first single-stranded oligonucleotide comprising a sequencing adaptor sequence and a primer sequence under conditions suitable for annealing of the first single-stranded oligonucleotide to the single stranded polynucleotide of step a., and then extending the primer sequence with a polymerase to produce double-stranded polynucleotide;
c. Denaturing the double-stranded polynucleotide of step b. to produce single stranded polynucleotide;
d. Incubating the single stranded polynucleotide from step c. with a second single-stranded oligonucleotide comprising a sequencing adaptor sequence and a primer sequence under conditions suitable for annealing of the second single-stranded oligonucleotide to the single stranded polynucleotide of step c., and then extending the primer sequence with a polymerase to produce double-stranded polynucleotide.

In any of the methods described herein, “denaturing” may be a step of disrupting hydrogen bonds that exist between nucleotides within polynucleotide and thus produce single stranded polynucleotide. Polynucleotide present in the sample to be applied to the method of the invention may be denatured to produce a single stranded polynucleotide. For example, where the polynucleotide is DNA, the DNA may be denatured in any way that the user deems appropriate. Denaturation may be performed chemical or heat treatment for any duration that the user deems appropriate. The DNA may be denatured using any alkaline denaturation method known in the art, for example, by subjecting the DNA to sodium hydroxide (NaOH) or potassium hydroxide (KOH), high salt conditions, or treatment with urea. Preferably, the DNA is denatured by subjecting the DNA to heat treatment. Preferably the heat treatment is short. Even more preferably, the heat treatment is at 95° C. for 1 minute.

In any of the methods described herein, the single stranded polynucleotide may be incubated with a first single-stranded oligonucleotide comprising a sequencing adaptor sequence and a primer sequence under conditions suitable for annealing of the first single-stranded oligonucleotide to the single stranded polynucleotide. The sequencing adaptor sequence may be 5′ to the primer sequence or 3′ to the primer sequence within the first single-stranded oligonucleotide. Preferably, the sequencing adaptor sequence is orientated 5′ to the primer sequence within the first single-stranded oligonucleotide. Primer sequences suitable for use in the methods described herein may comprise sequences specific to one or more targets, random sequences, partially random sequences, and combinations thereof. “Specific” in this context refers to conventional Watson-Crick base-pairing. Thus a first single-stranded oligonucleotide of sequence 5′-ACGA-3′ may hybridise to the single stranded polynucleotide of sequence 5′-TCGT-3′ wherein the G of the single-stranded oligonucleotide will be positioned opposite the C of the single stranded polynucleotide and will hydrogen bond therewith. This principle applies to any complementary oligonucleotide relationship disclosed herein, including oligonucleotides comprising universal nucleotides.

Reaction conditions suitable for the annealing, i.e. the hybridization of a nucleotide sequence to a complementary nucleotide sequence, of primer sequences to polynucleotides such as DNA and RNA are known in the art. The nucleotide composition of the primer sequence may be specific to a region of interest within the polynucleotide contained within the sample or may be random. The random nature of the oligonucleotide composition leads to random priming of the single stranded polynucleotide in the sample. Random priming of the first single-stranded oligonucleotide enables polymerase-mediated extension at random loci throughout the single stranded polynucleotide in the sample. In any of the steps of the method described herein involving an “extension” step, extension from the randomly primed first single-stranded oligonucleotides may be mediated through the use of a polymerase.

In any of the methods described herein, the polynucleotide may be RNA and the polymerase used to for extension from the primed first single-stranded oligonucleotides may be a reverse transcriptase. The reverse transcriptase produces a DNA strand that is complementary (cDNA) to the RNA polynucleotide. Many reverse transcriptases are known in the art, and the user may use any reverse transcriptase that they deem appropriate.

In any of the methods described herein, the polynucleotide contained in the sample may be DNA and the polymerase may be a DNA-directed DNA polymerase. Many DNA-directed DNA polymerases are known in the art, and the user may use any DNA-directed DNA polymerase that they deem appropriate. The DNA-directed DNA polymerase used may, for example, be a Klenow polymerase, a Vent polymerase, a Deep Vent polymerase, DNA Polymerase I or a T4 DNA Polymerase. Preferably, the Klenow, Vent and Deep Vent polymerases retain their exonuclease activity. The first single-stranded oligonucleotide primed to the ssDNA may be extended to synthesize a polynucleotide molecule comprising DNA or RNA, preferably DNA, that is complementary to the ssDNA in the sample. In the extensions steps described herein, nucleotides incorporated into the newly synthesized polynucleotide by the DNA-directed DNA polymerase may be a deoxynucleotide triphosphate (dNTP), such as dATP, dTTP, dCTP or dGTP, or a modified dNTP such as a modified dATP, a modified dTTP, a modified dCTP, a modified dGTP and/or a universal nucleotide. Any one or more of these nucleotides may be comprised within a reaction mixture with DNA-directed DNA polymerase. Other potential components of a DNA-directed DNA polymerase reaction mixture are well known in the art. A first double-stranded DNA (dsDNA) may be produced by extending the primer sequence that is annealed to the ssDNA in accordance with the invention described herein.

Priming and extension according to the methods described herein has the advantage over pre-existing methods by the fact that it maintains the integrity of potentially damaged polynucleotide in the sample. Other methods require a fragmentation step prior to incorporating sequencing adaptor sequences. Fragmentation methods such as sonication are known to potentially compromise the integrity of polynucleotide. Polynucleotide that is extracted from FFPE-treated tissue is often already damaged, fragmented and single-stranded, hence priming and extension maintains the integrity of potentially damaged polynucleotide in the sample.

The sequencing adaptor contained within the first single-stranded oligonucleotide of the invention may comprise any oligonucleotide sequencing adaptor known in the art. Exemplary sequencing adaptors are Illumina® sequencing adaptors that may be used with an Illumina® sequencing platform. Illumina sequencing adaptors are designed to be complementary to sequences that coat an Illumina sequencing flow cell, thus enabling adherence of sample polynucleotide to a flow cell and implementation of sequencing by synthesis and determination of the polynucleotide sequences in the sample.

In any of the methods described herein, the first single stranded polynucleotide may be denatured to produce the second single stranded polynucleotide. The first double stranded polynucleotide may be denatured in any way that the user deems appropriate. For example, denaturation may be performed chemical or heat treatment for any duration that the user deems appropriate. For example, denaturation may be performed chemical or heat treatment for any duration that the user deems appropriate. The first double stranded polynucleotide may be denatured using any alkaline denaturation method known in the art, for example, by subjecting the first double stranded polynucleotide to sodium hydroxide (NaOH) or potassium hydroxide (KOH), high salt conditions, or treatment with urea. Preferably, the first double stranded polynucleotide is denatured by subjecting the first double stranded polynucleotide to heat treatment. Preferably the heat treatment is short. Even more preferably, the heat treatment is at 95° C. for 1 minute.

In any of the methods described herein, the second single-stranded polynucleotide may be incubated with a second single-stranded oligonucleotide comprising a sequencing adaptor sequence and a random primer sequence under conditions suitable for annealing of the second single-stranded oligonucleotide to the second single-stranded polynucleotide. The sequencing adaptor sequence may be 5′ to the primer sequence or 3′ to the primer sequence within the second single-stranded oligonucleotide. Preferably, the sequencing adaptor sequence is orientated 5′ to the primer sequence within the second single-stranded oligonucleotide. Primer sequences suitable for use in the methods described herein may comprise sequences specific to one or more targets, random sequences, partially random sequences, and combinations thereof “Specific” in this context refers to conventional Watson-Crick base-pairing. Thus a second single-stranded oligonucleotide of sequence 5′-ACGA-3′ may hybridise to the ssDNA of sequence 5′-TCGT-3′ wherein the G of the single-stranded oligonucleotide will be positioned opposite the C of the second single-stranded polynucleotide and will hydrogen bond therewith. This principle applies to any complementary oligonucleotide relationship disclosed herein, including oligonucleotides comprising universal nucleotides.

Reaction conditions suitable for the annealing, i.e. the hybridization of a nucleotide sequence to a complementary nucleotide sequence, of primer sequences to polynucleotides are known in the art. The nucleotide composition of the primer sequence may be specific to a region of interest within the polynucleotide contained within the sample or may be random. The composition of the primer sequence is preferably random. The random nature of the oligonucleotide composition leads to random priming of the second single stranded oligonucleotide in the sample. Random priming of the second single-stranded oligonucleotide enables polymerase-mediated extension at random loci throughout the second single stranded oligonucleotide in the sample. In any of the steps of the method described herein involving an “extension” step, extension from the randomly primed second single-stranded oligonucleotides may be mediated through the use of a polymerase.

In any of the methods described herein, the second single-stranded polynucleotide may be DNA and the polymerase may be a DNA-directed DNA polymerase. Many DNA-directed DNA polymerases are known in the art, and the user may use any DNA-directed DNA polymerase that they deem appropriate. The DNA-directed DNA polymerase used may, for example, be a Klenow polymerase, a Vent polymerase, a Deep Vent polymerase, DNA Polymerase I or a T4 DNA Polymerase. Preferably, the Klenow, Vent and Deep Vent polymerases retain their exonuclease activity. The second single-stranded oligonucleotide primed to the second ssDNA may be extended to synthesize a polynucleotide molecule comprising DNA or RNA, preferably DNA, that is complementary to the second ssDNA in the sample. In the extensions steps described herein, nucleotides incorporated into the newly synthesized polynucleotide by the DNA-directed DNA polymerase may be a deoxynucleotide triphosphate (dNTP), such as dATP, dTTP, dCTP or dGTP, or a modified dNTP such as a modified dATP, a modified dTTP, a modified dCTP, a modified dGTP and/or a universal nucleotide. Any one or more of these nucleotides may be comprised within a reaction mixture with DNA-directed DNA polymerase. Other potential components of a DNA-directed DNA polymerase reaction mixture are well known in the art. A second dsDNA may be produced by extending the primer sequence that is annealed to the second ssDNA in accordance with the invention described herein.

Random priming and extension according to the methods described herein has the advantage over pre-existing methods by the fact that it maintains the integrity of potentially damaged polynucleotide in the sample. Other methods require a fragmentation step prior to incorporating sequencing adaptor sequences. Fragmentation methods such as sonication are known potentially compromise the integrity of polynucleotide. Polynucleotide that is extracted from FFPE-treated tissue is often already damaged, fragmented and single-stranded, hence random priming and extension maintains the integrity of potentially damaged polynucleotide in the sample.

The sequencing adaptor contained within the second single-stranded oligonucleotide of the invention may comprise any oligonucleotide sequencing adaptor known in the art. Exemplary sequencing adaptors are Illumina® sequencing adaptors that may be used with an Illumina® sequencing platform. Illumina sequencing adaptors are designed to be complementary to sequences that coat an Illumina sequencing flow cell, thus enabling adherence of sample polynucleotide to a flow cell and implementation of sequencing by synthesis and determination of the polynucleotide sequences in the sample.

In any of the methods described herein, the primer sequence in the first single-stranded oligonucleotide and/or the primer in the second single-stranded oligonucleotide is:
i. a random primer sequence, optionally comprising a random nonamer oligonucleotide sequence; or
ii. a primer sequence specific to a region of interest in the polynucleotide, optionally comprising a 20 mer oligonucleotide sequence.

In any of the methods described herein, wherein the primer sequence in the first single-stranded oligonucleotide of the invention is a primer sequence specific to a region of interest in the polynucleotide, optionally comprising a 20 mer oligonucleotide sequence, the primer sequence in the second single-stranded oligonucleotide of the invention is preferably a random primer sequence, optionally comprising a random nonamer oligonucleotide sequence.

In any of the methods described herein, wherein the primer in the first single-stranded oligonucleotide of the invention is a primer sequence specific to a region of interest in the polynucleotide, and the primer in the second single-stranded oligonucleotide of the invention is a random primer sequence, the sequencing adaptor sequence comprised within the second single stranded oligonucleotide preferably determines that sequencing on any suitable sequencing apparatus begins at the end of the double stranded polynucleotide comprising said sequencing adaptor sequence. This is particularly advantageous because beginning sequencing from a randomly primed and extended site maintains a high level of sequence diversity during the first sequencing cycles, thereby reducing the risk of low sequencing yield or low data quality. As described herein, any suitable sequencing techniques may be employed to determine the sequence of the DNA

In any of the methods described herein, wherein the primer sequence in the first single-stranded oligonucleotide and/or the primer in the second single-stranded oligonucleotide is a primer sequence specific to a region of interest in the polynucleotide, a plurality of first and/or second single stranded oligonucleotides may be used in order maximise coverage of the region of interest. Preferably, the plurality of first and/or second single stranded oligonucleotides comprises about 5 oligonucleotides per 1 kb of the region of interest, more preferably about 10 oligonucleotides per 1 kb of the region of interest, and even more preferably about 15 oligonucleotides per 1 kb of the region of interest. Most preferably, the plurality of first and/or second single stranded oligonucleotides are approximately evenly spaced across the region of interest.

In any of the methods described herein, the sequencing adaptor sequence of the first and/or second single-stranded oligonucleotide may include one or more of:
a sequence complementary to a sequencing primer sequence;
a sequence complementary to an amplification primer sequence;
a barcode or index sequence; and/or
a sequence to facilitate attachment to a solid surface, optionally wherein said sequence is complementary to an oligonucleotide attached to said surface.

A “sequence complementary to sequencing primer sequence” as used herein may be an oligonucleotide sequence which may be a complementary to a known primer sequence, thus enabling targeted sequencing sanger sequencing, or any other sequencing technology, for example high-depth high-throughput sequencing. A “sequence complementary to a sequencing primer sequence” may also perform the same function as sequencing adaptor sequences within the first and/or second single-stranded oligonucleotide of the methods described herein by being of complementary sequence to that of sequencing adaptor sequences that coat an Illumina flow cell, thus enabling adherence of sample polynucleotide to a flow cell and implementation of sequencing by synthesis and determination of the polynucleotide sequences in the sample. A “sequence complementary to an amplification primer sequence” as used in the methods described herein may particularly be used to amplify all, or targeted regions, of sample polynucleotide prior to sequencing. Amplification of all, or targeted regions, of sample polynucleotide may be particularly useful and effective for low quantities of input polynucleotide in the methods of the invention described herein. In the methods described herein, “barcode sequence” and “index sequence” may be used interchangeably. An “index sequence” may also perform the same function as a sequence complementary to an amplification primer sequence within the first and/or second single stranded oligonucleotide. Preferably, a “index sequence” may preferably be used to multiplex samples and/or polynucleotide sequencing libraries. Indexing samples and/or polynucleotide sequencing libraries enables multiples samples and/or libraries to be pooled and sequenced together. Indexing may be applied in a “single” or “dual” indexing manner, and methods for such indexing techniques are well known in the art. The methods of the invention described herein are suitable for large scale multiplexing of both library preparation and sequencing. A first and/or second single-stranded oligonucleotide of the methods described herein, whilst not being limited to these sequences, and whilst not being limited to any particular orientation of these sequences within a first and/or second single-stranded oligonucleotide, may comprise any one of, or a plurality of, the following sequences:
a sequencing adaptor sequence
a primer sequence
a sequence complementary to an amplification primer sequence
a barcode or index sequence and/or
a sequence to facilitate attachment to a solid surface, optionally wherein said sequence is complementary to an oligonucleotide attached to said surface.

In the methods of the invention described herein, the extension step, i.e. following the annealing of a first or second single stranded oligonucleotide comprising a sequencing adaptor and a primer sequence to a single stranded polynucleotide, may be conducted by incubating the polymerase with a suitable reaction mixture at approximately 4° C., before slowly increasing the temperature up to the optimal operating temperature of the polymerase and holding at said optimal operating temperature until extension is substantially complete. In any of the methods described herein, the polynucleotide may be DNA and the polymerase may be a DNA-directed polymerase. In any of the methods described herein, the polynucleotide may be RNA and the polymerase used to for extension from the primed first single-stranded oligonucleotides may be a reverse transcriptase.

The extension reaction may first be incubated at 4° C. for at least about 1 minute, at least about 2 minutes, at least about 3 minutes, at least about 4 minutes, at least about 5 minutes, at least about 6 minutes, at least about 7 minutes, at least about 8 minutes, at least about 9 minutes, or at least about 10 minutes. Preferably, the extension reaction is first incubated at 4° C. for approximately 5 minutes. In this step of the methods described herein, temperature is slowly increased up to the optimal temperature of the DNA-directed DNA polymerase before holding at said optimal operating temperature until extension is substantially complete. A slow ramping rate (rate of increase in temperature from 4° C. up to the optimal temperature of the polymerase) is preferable in the methods described herein. The ramping rate may be no more than around 1° C./minute, no more than around 2° C./minute, no more than around 3° C./minute, no more than around 4° C./minute, no more than around 5° C./minute, no more than around 6° C./minute, no more than around 7° C./minute, no more than around 8° C./minute, no more than around 9° C./minute, no more than around 10° C./minute, no more than around 20° C./minute, no more than around 30° C./minute, no more than around 40° C./minute, no more than around 50° C./minute or no more than around 100° C./minute. The optimal operating temperature of the specific polymerase used will vary depending on the polymerase used. For example, many DNA-directed DNA polymerases are known in the art, and the user may use any DNA-directed DNA polymerase that they deem appropriate. The DNA-directed DNA polymerase used may, for example, be a Klenow polymerase, a Vent polymerase, a Deep Vent polymerase, DNA Polymerase I or a T4 DNA Polymerase. Preferably, the Klenow, Vent and Deep Vent polymerases retain their exonuclease activity. Preferably, the optimal operating temperature of the DNA-directed DNA polymerase is around 37° C. and the temperature is increased to this temperature at a rate of no more than around 4° C./minute, Preferably, the DNA-directed DNA polymerase is Klenow polymerase.

In any of the methods described herein, the second double-stranded polynucleotide may be amplified in order to produce copies of the second double-stranded polynucleotide in the sample. The amplification step may involve polymerase chain reaction (PCR). The amplification step may involve the use of primer sequences complementary to at least part of the sequencing adaptor sequences introduced to the double-stranded polynucleotide in the methods of the invention described herein. For example, when Illumina® sequencing adaptors have been used in the methods of the invention described herein, primer sequences comprising complementary nucleotide sequences to at least part of the Illumina® adaptor sequences may be used in the PCR reaction. PCR may be performed under conditions known in the art and at temperatures suitable for efficient annealing of the primer sequences. PCR may be optimised to reduce GC bias and prevent incorporation of errors into the copies of the DNA in the sample. The second dsDNA may be amplified by PCR using less than 40 cycles. The second dsDNA may be amplified by PCR using less than 30 cycles. The second dsDNA may be amplified by PCR using less than 20 cycles. The second dsDNA may be amplified by PCR using less than 10 cycles. The second dsDNA may be amplified by PCR using less than 5 cycles. The second dsDNA may be amplified by PCR using 2, 3, 4, 5, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 cycles. Preferably, the second dsDNA is amplified by PCR using 10 cycles. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA).

In the methods of the invention described herein, one or more steps of the method may comprise extraction of polynucleotide from a sample. Polynucleotide comprised within a sample to be applied to the methods described herein may require extraction prior to denaturation. The method of extraction depends on the material within which polynucleotide is comprised. Furthermore, the method of extraction may depend on what type of polynucleotide is contained within the sample e.g. DNA or RNA. Methods of extracting DNA from, for example, hair and hair follicles, blood and other biohumoral fluids, animal tissue, soil, and cells are well known in the art.

In the methods of the invention described herein, the sample may comprise polynucleotide with damaged nucleotide bases. Nucleotide bases may, for example, be damaged as a result of deamination, oxidation, depurination, depyrimidination. In the methods of the invention described herein, one or more steps of the method may comprise removing damaged bases from the polynucleotide with at least one base excision repair enzyme.

Base excision repair enzymes can be applied to single-stranded polynucleotide or double-stranded polynucleotide in the methods described herein. Any suitable base excision repair enzyme may be used depending on what type of polynucleotide is contained within the sample e.g. DNA or RNA. In any of the methods described herein, one or more base excision repair enzymes may be used in the steps of the method comprising removing damaged bases from the polynucleotide. Steps of the method comprising a base excision repair step may comprise removing damaged bases from the polynucleotide and replacement of damaged bases with undamaged bases. In other instances, steps of the method comprising a base excision repair step may comprise removing damaged bases from the polynucleotide without replacement of the damaged bases with undamaged bases. The base excision repair enzyme may be any suitable base excision repair enzyme known in the art. Exemplary base excision repair enzymes for subjecting to single-stranded DNA or double-stranded DNA comprise APE 1, Endo III, TMA Endo III, Endo IV, Tth Endo IV, Endo V, Endo VIII, Fpg, hOGG1, hNEIL1, hNEIL2, hNEIL3, T7 Endo I, T4 PDG, UDG & Afu UDG, Afu UDG, SMUG1, hAAG. The base excision repair enzyme is preferably a glycosylase enzyme, or more preferably any one or more of hNEIL1, hNEIL2, hNEIL3, Fpg or SMUG1. Even more preferably the base excision repair enzyme is SMUG1 and/or Fpg.

The methods described herein may further comprise removal of any remaining single stranded oligonucleotides that are not annealed to the first or second single-stranded polynucleotide for the purpose of polynucleotide extension. Short single-stranded polynucleotide fragments may also be removed. A “short” fragment may refer to any single stranded polynucleotides that are shorter that of the single stranded oligonucleotides used in the context of the invention. The removal of any remaining single stranded oligonucleotides and/or short single-stranded polynucleotide fragments may be performed by any suitable method known in the art. For example, the methods described herein may further comprise removal of any remaining single stranded oligonucleotide and/or short single-stranded polynucleotide fragments with an exonuclease. Preferably, the exonuclease is a nuclease with 3′ to 5′ activity, or with 5′ to 3′ activity, or with both 3′ to 5′ activity and 5′ to 3′ activity. Exemplary exonucleases include Lamda Exonuclease, RecJ, Exonuclease II, Exonuclease I, Thermolabile Exonuclease I, Exonuclease T, Exonuclease V (RecBCD), Exonuclease VIII truncated, Exonuclease VII, Nuclease BAL-31, T5 Exonuclease, T7 Exonuclease. Preferably, the nuclease is any nuclease known in the art with 3′ to 5′ activity. Even more preferably, the exonuclease is Exonuclease I (NEB).

Preferably, a step removal of any remaining single stranded oligonucleotides and/or short single-stranded polynucleotide fragments is applied to the methods of the invention after the step of producing the first double-stranded polynucleotide and prior to the step of denaturing the first double-stranded polynucleotide. Alternatively, the first double-stranded polynucleotide may be purified after the step of producing the first dsDNA and prior to the step of denaturing the first double-stranded polynucleotide. Further alternatively, after the step of producing the first double-stranded polynucleotide and prior to the step of denaturing the first double-stranded polynucleotide, a step removal of any remaining single stranded oligonucleotides and/or short ssDNA fragments is applied to the methods of the invention and the first double-stranded polynucleotide may be purified. Preferably, the second double-stranded polynucleotide may be purified after the step of producing the second double-stranded polynucleotide.

In methods described herein, steps involving purifying double-stranded polynucleotide can be performed by any methods known in the art that are suitable for purifying double-stranded polynucleotide. Depending on the type of polynucleotide contained in the sample, different known polynucleotide purification methods may be more suitable. Exemplary methods for purifying DNA include organic extraction methods such as ethanol precipitation or phenol-chloroform precipitation, Chelex extraction purification, and solid phase purification, and any known DNA purification kits in the art. Preferably, purification steps to be used in the methods described herein use solid phase reversible immobilization (SPRI) beads.

In methods of the invention described herein, wherein the primer in the first single stranded oligonucleotide is a primer sequence specific to a region of interest within a polynucleotide, it is preferable for the method to comprise removal of any remaining single-stranded oligonucleotides, and optionally short single-stranded polynucleotides, following annealing of the first single stranded oligonucleotide to the single polynucleotide and prior to extending the primer with a polymerase to produce double-stranded polynucleotide. Removal of any remaining single stranded oligonucleotides may be achieved by purifying single stranded polynucleotide that is annealed to the first single stranded oligonucleotide. Exonuclease digestion may then be performed to remove any remaining single stranded oligonucleotide and/or short single-stranded polynucleotide. Further optionally, additional cycles of (i) purifying single stranded oligonucleotide that is annealed to the first single stranded oligonucleotide; and/or (ii) exonuclease digestion; may be performed prior to extending the primer with a polymerase to produce double-stranded polynucleotide. Preferably, in any of the methods described herein wherein the primer in the first single stranded oligonucleotide is a primer sequence specific to a region of interest within a polynucleotide, following the denaturing of the polynucleotide in the sample and the annealing of the first single-stranded oligonucleotide, it is preferable for the method to comprise:

i. removal of any remaining first single-stranded oligonucleotides by purification of the single-stranded polynucleotide that is annealed to the first single stranded oligonucleotide;

ii. digestion of any remaining first single-stranded oligonucleotide with an exonuclease;

iii. further purification of the single-stranded polynucleotide that is annealed to the first single stranded oligonucleotide.

Further preferably, in any of the methods described herein wherein the primer in the first single stranded oligonucleotide is a primer sequence specific to a region of interest within a polynucleotide, following the denaturing of the polynucleotide in the sample and the annealing of the first single-stranded oligonucleotide, it is preferable for the method to comprise:

i. removal of any remaining first single-stranded oligonucleotides by purification of the single-stranded polynucleotide that is annealed to the first single stranded oligonucleotide using SPRI beads;

ii. digestion of any remaining first single-stranded oligonucleotide with Exonuclease I;

iii. further purification of the single-stranded polynucleotide that is annealed to the first single stranded oligonucleotide using SPRI beads.

The methods described herein may further comprise a step of sequencing the population of DNA molecules generated by the methods of the invention described herein. The step of sequencing the DNA may be for the purposes of determining its entire, or a portion of, its sequence. Any suitable sequencing techniques may be employed to determine the sequence of the DNA. In the methods of the present invention, the use of high-throughput, so-called “second generation”, “third generation” and “next generation” techniques may be used to sequence the DNA.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, Calif.; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore (https://www.nanoporetech.com/). In an alternative method, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### Example 1

As described herein, it was surprisingly found that adapting methods previously developed for DNA methylation analysis permits the circumvention of several inefficient steps associated with pre-existing adaptor ligation-based library preparation methods, resulting in the improved library preparation methods of the invention. Degraded DNA adaptor tagging (DDAT) is an exemplary method of the invention described. DDAT utilises random priming which can amplify single stranded ssDNA in addition to dsDNA that is captured by current commercially available kits. In this study, the DDAT method is compared to a standard preparation method that utilises adaptor ligation, with each method being evaluated for library quality and yield when used on FFPE samples of varying quality. The DDAT method is found to be particularly effective.

### Materials and Methods

### Sample Information

Samples were obtained from the University College London Hospitals Biobank (REC: 15/YH/0311) the Oxford University Hospitals (MREC 10/H0604/72).

### Genomic DNA Extraction

DNA was extracted from formalin fixed paraffin embedded (FFPE) colorectal cancer samples using the High Pure FFPET DNA isolation kit (Roche Diagnostics Ltd.) according to the manufacturer's protocol. DNA was quantified using the Qubit® 3.0 fluorometer (Life Technologies) and quality was estimated using a multiplex PCR-based assay as previously described.

### Whole Genome Sequencing (WGS) Library Preparation (Degraded DNA Adaptor Tagging Protocol)

To remove damaged bases, 2 ng of good or poor quality FFPE DNA and 10 ng of very poor quality DNA was combined with 5 U of SMUG1, 1 U Fpg, 1×NEB buffer 1 and 0.1 μg/ml BSA (NEB) in 10 μl and incubated for 1 hr at 37° C. (This enzyme digestion step was excluded in the pilot experiment). First strand synthesis was performed immediately afterwards by combining the 10 μl reaction with 1× blue buffer, 400 nM dNTPs and 4 uM oligo 1 (5′-CTACACGACGCTCTTCCGATC-3′) (SEQ ID NO: 1, and ‘N’ can be any nucleotide) in 49 μl. Samples were heating to 95° C. for 1 min and immediately cooled on ice. 50 U of Klenow exo-; Enymatics) fragment was added to each sample and the tubes were incubated at 4° C. for 5 min before slow ramping (4° C./min) to 37° C. (i.e. 8 minutes for the ramping step), then held at 37° C. for 90 minutes. After this step samples can be stored overnight at −20° C. if required. The remaining primers were digested with 20 U of exonuclease I (NEB) at 37° C. for 1 hr in 100 μl before purification using AMPure XP beads (Beckman). For purification, 80 μl AMPure XP beads were added directly to the samples and incubated for 10 minutes at room temperature. After collecting beads on a magnet we performed 2×200 μl 80% ethanol washes on the magnet. Beads were dried for 6-10 min being vigilant not to allow beads to over dry and crack. DNA was eluted in 38 μl of water before adding components for second strand synthesis (1× blue buffer, 400 nM dNTPs and 0.8 μM oligo 2 (5′-CAGACGTGTGCTCTTCCGATCT-3′) (SEQ ID NO: 2, and ‘N’ can be any nucleotide)) to the PCR tube still containing the beads. Samples were heated at 98° C. for 2 min then incubated on ice before 50 U of Klenow exo-) was added and incubated using the same conditions as for first strand synthesis. To purify the second strand synthesis reaction, an aliquot of AMPure XP beads was centrifuged and the supernatant collected. After addition of 50 μl of water to the sample, 80 μl of bead buffer was added and mixed to resuspend the beads still within the tube and the DNA was purified as described above. After the final drying step, beads were resuspended in 33 μl of water and incubated for 10 min to elute the DNA. The beads were collected using a magnetic rack and the 33 μl of purified DNA transferred to a new PCR tube before adding the components for the final library PCR amplification (lx KAPA HiFi buffer, 400 nM dNTPs, 1 U KAPA HiFi Hotstart Taq, PE1.0

(SEQ ID NO: 3)
(5′-AATGATACGGCGACCACCGAGATCTACACTCTTTCCCTACACGAC
GCTCTTCCGATCT-3′)

and the indexed custom reverse primer based on the Illumina TruSeq sequence (5′-CAAGCAGAAGACGGCATACGAGATCGTGATGTGACTGGAGTTCAGACGTGTGCT CTTCCGATCT-3′) (SEQ ID NO: 4). For the pilot experiment, the library was dual indexed using NEBNext® Multiplex Oligos for Illumina® (NEB). Samples were amplified for 10 PCR cycles before purification of library using a 1:0.8 ratio of DNA to beads and elution in 15 μl of water. The library was quantified using the Qubit® 3.0 fluorometer, 2200 TapeStation (Agilent, Santa Clara, Calif.) and KAPA Library Quantification Kit (Roche).

### Whole Genome Sequencing (WGS) Library Preparation (Standard Protocol)

FFPE DNA was sonicated using the Covaris M220 focused-ultrasonicator to an average fragment size of 300 bp. DNA was then repaired using the NEBNext® FFPE DNA Repair Mix, according the manufacturer's protocol (New England Biolabs, Hitchin, UK). Library preparation was performed using the NEBNext® Ultra™ DNA Library Prep Kit for Illumina® according to the manufacturer's protocol for FFPE samples (New England Biolabs; half volumes of all reagents were used in the pilot experiment) and 10 cycles of library amplification, during which the library was indexed using custom PE1.0 (5′-AATGATACGGCGACCACCGAGATCTACACTCTTTCCCTACACGACGCTCTTCCGA TCT-3′) (SEQ ID NO: 3) and indexed reverse primer (5′-CAAGCAGAAGACGGCATACGAGATCGTGATGTGACTGGAGTTCAGACGTGTGCT CTTCCGATCT-3′ (SEQ ID NO: 4); index sequence underlined). For the pilot experiment, the library was dual indexed using NEBNext® Multiplex Oligos for Illumina® (NEB). The library was quantified using the same methods as described for DDAT.

### Sequencing Analysis and Bioinformatics Analysis Pipeline

For each sample, the paired-end sequence reads were initially quality checked with FastQC v0.11.5 to investigate base quality scores, sequence length distributions, and additional features of the data. The reads were then aligned to the reference human genome hg19 (for the pilot experiment) and hg38 (for the samples in Table 1) by the BWA-MEM algorithm used in Burrows-Wheeler Aligner (BWA) v0.7.8. The resulting SAM file was processed into a BAM file using Samtools v1.3.1, then sorted and indexed with PCR duplicates marked using Picard v2.6 and v2.12 (for the pilot experiment and samples in Table 1, respectively). The final BAM files were quality checked using BamQC v0.1 and Picard v2.12 to investigate mapping qualities, percentage of soft clipped reads, and other basic statistics of the processed data shown in Table 2 and Table 4. Coverage statistics and mapped insert size histograms (reads/base) were calculated with DepthofCoverage tool in GATK v3.6 and Picard v2.12. VCF files were generated using GATK v4.0 Mutect2.

### Statistical Analysis

Significance testing was performed using Prism (v.5.04) and one-way ANOVA with Bonferroni post-hoc tests as specified in the Figure legend. Where applicable, data are plotted as mean±SEM.

TABLE 1
Sample and preparation details for WGS comparing
standard vs. DDAT library preparation.

| | | | | | PCR cycles |
|---|---|---|---|---|---|
| | | | Damaged base | Input | for final |
| Sample | Preparation | Soni- | repair/removal | DNA | library |
| quality | method | cation | included? | (ng) | amplification |
| Good | Standard | Yes | Yes | 2 | 10 |
| Poor | Standard | Yes | Yes | 2 | 10 |
| Very poor | Standard | Yes | Yes | 10 | 10 |
| Good | DDAT | No | Yes | 2 | 10 |
| Poor | DDAT | No | Yes | 2 | 10 |
| Very poor | DDAT | No | Yes | 10 | 10 |
| Good | DDAT | No | No | 2 | 10 |
| Poor | DDAT | No | No | 2 | 10 |
| Very poor | DDAT | No | No | 10 | 10 |

### Results and Discussion

### DDAT Library Preparation Improves Sequencing Quality and Increases Depth Compared to Standard Methods

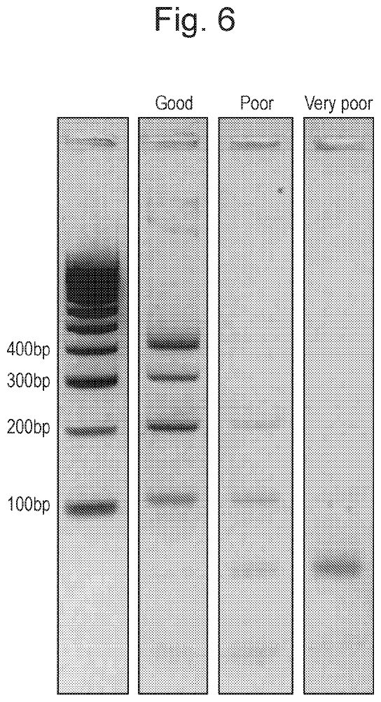

A pilot experiment was first performed to compare WGS data generated using the DDAT and the NEBNext Ultra II (‘standard’) library preparation methods, using a representative FFPE colorectal cancer DNA sample. It was anticipated that the DDAT method would be of greatest benefit for FFPE DNA which was substantially degraded, as these samples contain more ssDNA that is inaccessible using the standard method. Poor quality FFPE DNA was therefore used for the first test of the DDAT method (see FIG. 6 for assessment of FFPE DNA quality using multiplex PCR).

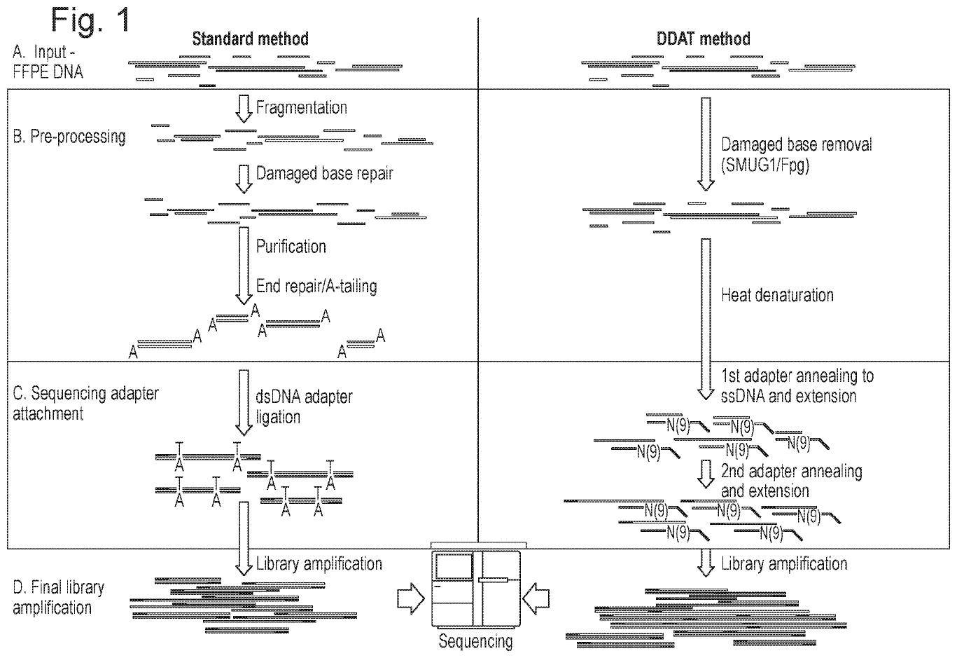

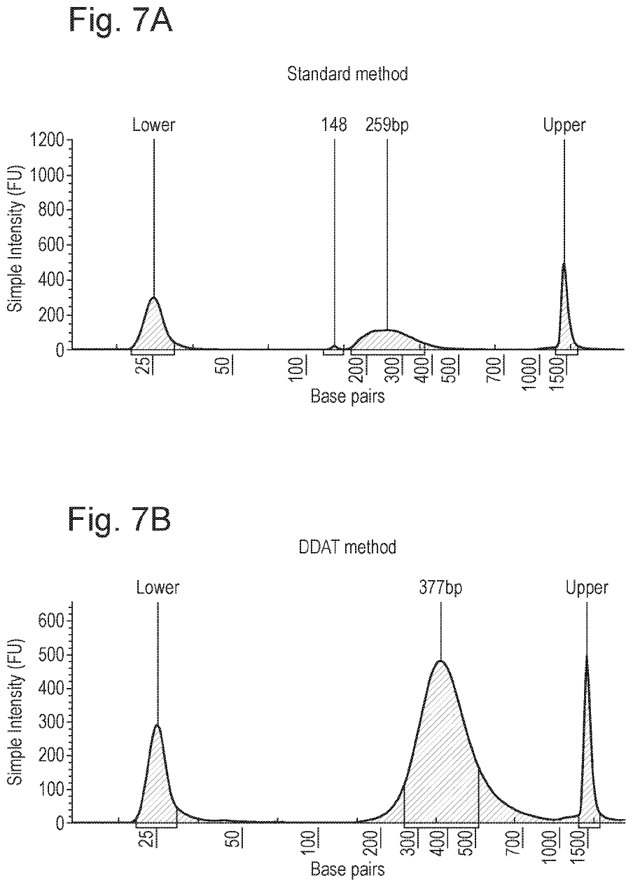

The DNA input for both library preparation methods was 2 ng and both used 10 PCR cycles of final library amplification. In the pilot experiment, the ‘Damaged base removal’ step in the DDAT method (FIG. 1) was not used. After preparing libraries and performing quality controls (FIG. 7, Table 3), samples were sequenced on Illumina's HiSeq X Ten, achieving close to 440 million raw reads in both cases.

TABLE 3
Sample metrics prior to sequencing for pilot
comparison of standard vs. DDAT method.

| Library | Concen- | Average | Concen- | | Total |
|---|---|---|---|---|---|
| preparation | tration | fragment | tration | Volume | quantity |
| method | (ng/μl) | size (bp) | (nM) | (μl) | (ng) |
| Standard | 0.71 | 259 | 7 | 15 | 10.65 |
| DDAT | 1.8 | 377 | 5 | 15 | 27 |

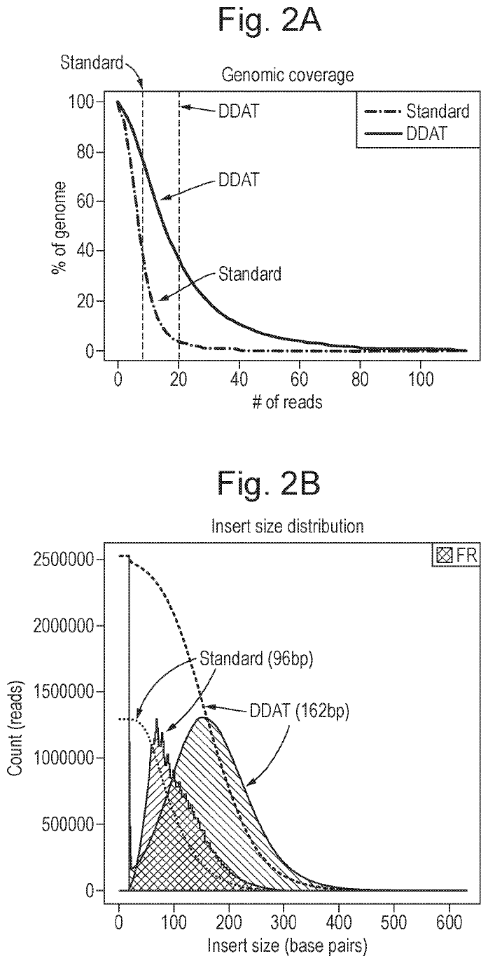

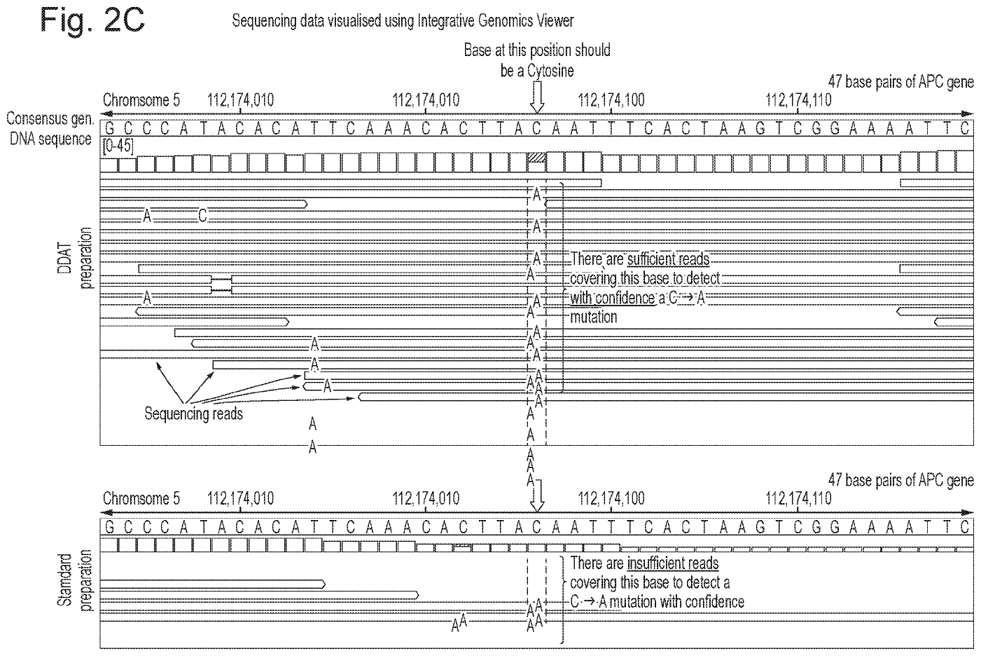

After filtering and mapping the reads to the human genome, the alignment metrics were assessed and found the DDAT method gave a mean 2.5-fold increase in coverage (FIG. 2A, Table 4) and 80% of these reads had a high mapping quality (MAPQ>=20) compared to 70% using the standard method (Table 4). The DDAT-generated library also had a larger median insert size of 162 bp compared to 96 bp (FIG. 2B), another indication of an improved library preparation, with the caveat that the standard preparation includes an initial fragmentation step by sonication which may explain this difference (see FIG. 1).

TABLE 4
WGS data alignment metrics from pilot comparison of
the standard vs. DDAT library preparation methods.

| | Standard | DDAT |
|---|---|---|
| Mean coverage (reads/base) | 8.14 | 20.26 |
| Unmapped sequences (% of | 7.168 | 6.506 |
| reads) | | |
| Soft clips (% of reads) | 35.924 | 14.219 |
| High mapping quality (MAPQ % | ~70 | ~80 |
| of reads) | | |

To illustrate the utility of improved library preparation when identifying putative driver mutations in human cancers, aligned reads were viewed on the Integrative Genome Viewer and a putative driver mutation (TAA) in the APC gene (p.Y935*, c.2805C>A, FIG. 2C). This mutation would be identified in the DDAT dataset using standard variant calling pipelines (altered reads=9, total reads=19, VAF=0.474), but would likely have been filtered out of the data produced by the standard method due to only two reads covering the base (altered reads=2, total reads=2, VAF=1). The pilot experiment showed that WGS data could be generated with greater clinical value from 2 ng of input DNA using DDAT compared to the standard method.

### The DDAT Protocol Improves Library Yield Compared to Standard Methods, and can be Used for Very Degraded FFPE Samples

To perform a more comprehensive comparison of the two WGS library preparation methods, the inventors selected three FFPE colorectal cancer DNA samples of variable quality (FIG. 6; samples highlighted by boxes). FFPE treatment of tissue commonly results in damage to DNA such as cytosine deamination to uracil. Removing and/or repairing damaged DNA bases can help to prevent false positive mutational calls in WGS data. Since the repair of the damaged base using commercially available kits is reliant on a complementary strand template (as is the case for the standard method), damaged bases within ssDNA cannot be repaired since there is no opposite strand. The inventors therefore assessed whether excision of the damaged base, without repair, would improve the quality of the WGS data from FFPE DNA. To do this, the DDAT protocol was modified to include an initial enzyme digestion step using commercially available SMUG1 (excises deoxyuracil and deoxyuracil-derivatives) and Fpg (an N-glycosylase and an AP-lyase which removes damaged based such as 8-oxoguanine). These enzymes create an abasic site in ssDNA and dsDNA, and the AP-lyase activity of Fpg creates a nick in the DNA backbone. In the standard method, a polymerase would then repair the gap in a dsDNA fragment by adding the missing complementary base; in contrast, in the DDAT method the missing base is not added and a heat denaturation separates the DNA strands, creating shorter ssDNA fragments where a damaged base has been removed. Table 1 summarises the experimental setup for this series of tests. The input quantity of the very poor quality sample was increased to 10 ng as the DNA was substantially degraded (Supplementary FIG. 1).

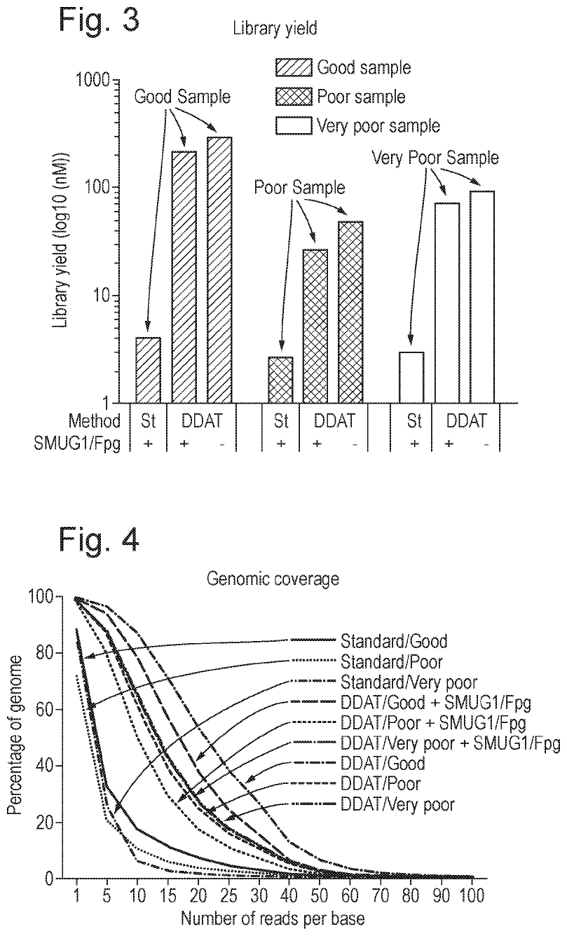

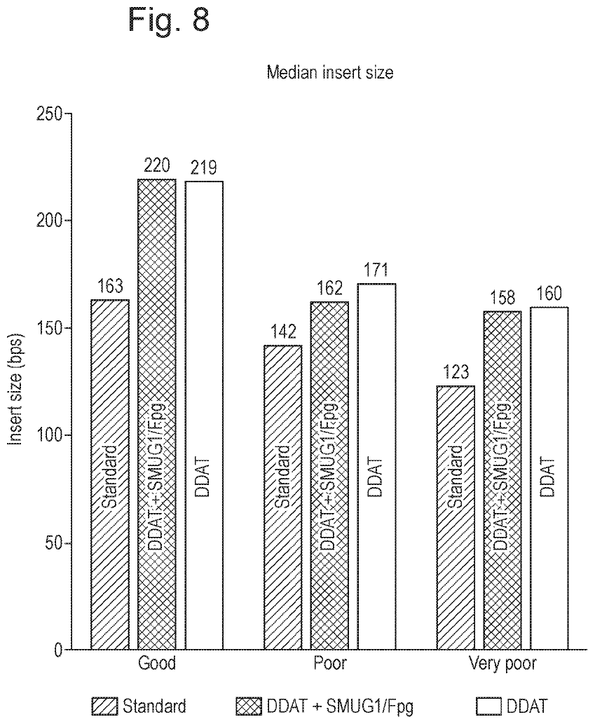

Total yield of each sequencing library was measured and it was found that the DDAT method (including damaged DNA removal) gave higher library yields compared to the standard method for all samples (FIG. 3; good: 52-fold, poor: 9.8-fold and very poor: 23-fold). The addition of the damaged base removal step caused a slight decrease in library yield of the DDAT method (good: 1.35-fold, poor: 1.8-fold and very poor: 1.3-fold). When assessing insert size, the DDAT method (with or without damaged base removal) gave higher median insert sizes for all samples compared to the standard method (FIG. 8) which indicates better library quality. In general, the increased library yields and insert size indicated that the DDAT method was capturing more of the input DNA compared to the standard method, validating the results of the pilot experiment.

### Genome Coverage for DDAT is Up to 3.7 Fold Higher Compared to the Standard Method

After sequencing the samples and aligning the reads to the human genome, the alignment metrics were assessed (Table 2). In general, data from the DDAT libraries were of higher quality than the standard libraries. The DDAT method resulted in higher mapping quality and lower proportions of chimeras and improper read pairs for all three samples. The addition of the damaged DNA removal step to the DDAT method did not have a consistent effect on the quality of the sequencing data, based on MAPQ scores. However, it is notable that in these samples the DDAT method resulted in a higher percentage of unmapped reads (see Conclusion).

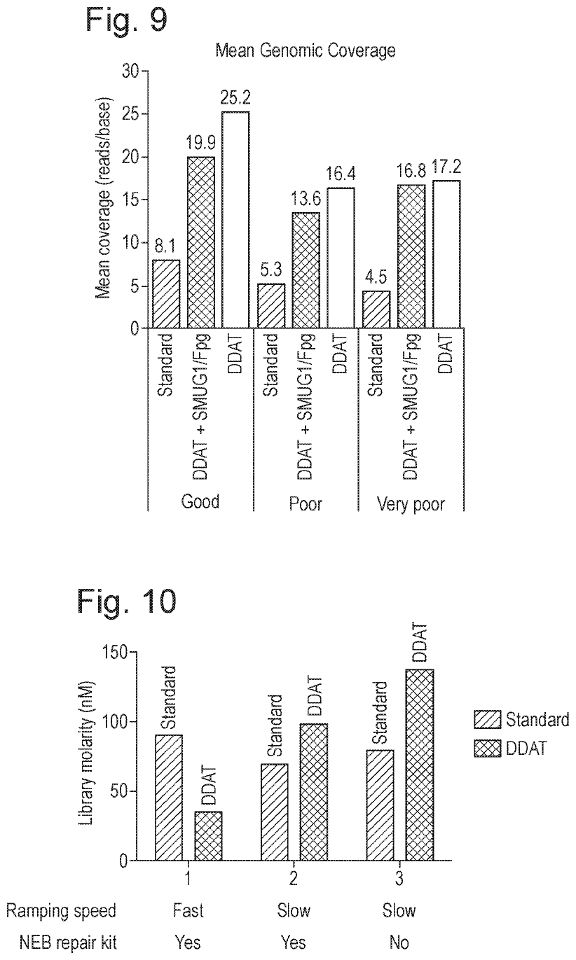

In agreement with the pilot experiment, the samples prepared using the DDAT method had higher genomic coverage than those prepared using the standard method (See FIG. 4, For DDAT+SMUG1/Fpg; good: 2.45-fold, poor: 2.54-fold, very poor: 3.77-fold; see FIG. 9). For the good and poor quality samples, adding the damaged base removal step in the DDAT method decreased the coverage achieved in the aligned reads (see FIG. 4 and FIG. 9); however, for the very poor sample, the coverage remained the same (see FIG. 4 and FIG. 9).

### Glycosylase Excision of Damaged DNA Bases Reduces FFPE-Induced Sequencing Artefacts in DDAT

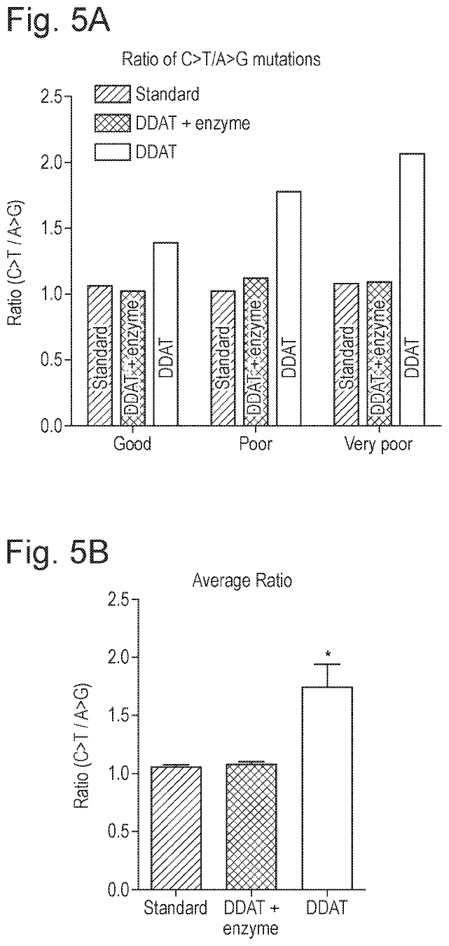

To quantify whether removing the damaged DNA bases using enzymes SMUG1 and Fpg decreased the number of sequencing artefacts in the DDAT method, the ratio of C>T/A>G transitions within each dataset was calculated (FIG. 5A). This showed that when the damaged DNA bases were removed, the ratio decreased, therefore, including the enzyme digestion step significantly decreases the presence of C>T transitions for all FFPE samples (FIG. 5B). This is comparable to the standard library preparation method which includes a DNA damage repair step. This demonstrates the importance of including SMUG1/Fpg digestion prior to the DDAT protocol to avoid FFPE-induced sequencing artefacts.

In summary, the DDAT library preparation method increases the library yield and quality of WGS data when compared to a standard method. Therefore, application of DDAT to sequencing of degraded FFPE samples is expected to recover a larger fraction of the starting DNA material than standard methods. This increases library yield, allowing for fewer PCR cycles prior to sequencing and therefore fewer PCR duplicates in the sequencing data and a 2- to 3-fold increase in genomic coverage. In addition, since the library yield is higher, a lower amount of input DNA can be used, saving precious clinical material. DDAT does not require DNA shearing or sonication as FFPE treatment in itself causes DNA fragmentation, and only a short heat step is required to denature the dsDNA rendering it accessible for random primer amplification. By using DDAT, samples considered not amplifiable with standard methods can be used to generate sequencing libraries of improved quality, and furthermore the per-sample cost of DDAT is lower than commercially available kits. In other words, for the same sequencing throughput, 3- to 4-fold more usable reads are produced. The quality of the DDAT sequencing data is dependent on inclusion of an enzyme digestion step to remove FFPE-induced damaged DNA bases, minimising FFPE-associated sequencing artefacts. Finally, the quality of the sequencing is significantly improved and, therefore, more robust biologically relevant information can be extracted.

### Conclusion

The inventors have established a new methodology for generating a population of DNA molecules, which optionally form a DNA sequencing library, using DDAT, which gives superior library yield and quality of WGS data from FFPE DNA compared to a standard commercially available kit. The improved efficiency is due to the two random priming and extension steps which enables ssDNA and dsDNA capture. As a result, the input DNA does not require an additional DNA fragmentation step (e.g. by sonication) before using DDAT, which further maintains the integrity of the DNA. This is particularly important when the input DNA is extracted from FFPE-treated tissue which is often already highly fragmented and single stranded.

During optimisation of the protocol the inventors discovered that the ramp rate used to reach the 37° C. incubation step during the first and second strand synthesis was important for efficient library preparation, with a faster ramping rate (132° C./min vs. 4° C./min) reducing the overall library yield (FIG. 10). The reason for this effect is unclear, however we hypothesize that the ramping rate affects the kinetics of random primer/DNA/Klenow binding, meaning that complexes are formed more efficiently if the temperature is gradually increased.

To detect the level of DNA degradation in our FFPE DNA the inventors used multiplex PCR of the GAPDH gene (FIG. 6), as this has been shown to give a good prediction of the quality of data from array comparative genomic hybridization for detecting CNVs, but further in depth assessment including a greater range of degraded FFPE samples is needed to establish how well multiplex PCR predicts the quality of WGS data.

The inventors have shown that removing damaged DNA bases in the DDAT method is sufficient to rescue the WGS data from FFPE-induced sequencing artefacts. Removal is the only option as the damaged bases in ssDNA cannot be repaired as there is no complementary strand to use as a template. Removal rather than repair does not seem to negatively impact the resulting WGS data as the yield and quality of data from the DDAT preparation with damaged base removal is generally improved compared to the standard method; furthermore, this type of damaged based removal has been shown to be effective for low DNA input targeted sequencing.

The inventors considered whether the DDAT method would have potential problems, similar to those recently identified when using the PBAT (post-bisulfite adapter tagging) method for whole genome bisulfite sequencing. Namely, that the random priming increases chimaeric reads (https://sequencing.qcfail.com/articles/pbat-libraries-may-generate-chimaeric-read-pairs/). However, based on the alignment statistics this does not appear to be the case when using DDAT as in fact the inventors observe a lower proportion of chimeric reads for DDAT prepared libraries than for standard libraries (Table 2).

Alternative methods exist that can utilise ssDNA as well as dsDNA for WGS, for example, a method for generating WGS libraries from ancient DNA, and for targeted sequencing from clinical samples. However, both these methods rely on ligation of a single stranded adapter to ssDNA, which is inefficient compared to the random priming used in DDAT and therefore will give inferior library yield and sequencing data from low quantities of input DNA.

In summary, the inventors have developed DDAT as an alternative WGS library preparation method which is particularly suited to highly degraded DNA samples containing ssDNA (e.g. archival FFPE samples). DDAT increases the yield and quality of FFPE WGS data and the inventors anticipate that this method can be applied to generate high quality WGS data from low input quantities, particularly from good quality starting material, improving the user's ability to obtain relevant data from samples previously deemed unsuitable for WGS.

### Example 2

As described herein, it was surprisingly found that adapting methods previously developed for DNA methylation analysis permits the circumvention of several inefficient steps associated with pre-existing adaptor ligation-based library preparation methods, resulting in the improved library preparation methods of the invention. Targeted DNA adaptor tagging (TDAT) is an exemplary method of the invention described. TDAT utilises targeted priming which can amplify single stranded DNA (ssDNA) and double stranded DNA (dsDNA), and thereby providing an advantage over commercially available kits which can only capture dsDNA. In this study, the TDAT method (which utilises targeted priming) was compared to the DDAT method (which utilises random priming), with each method being evaluated for the ability to detect genomic variants. The TDAT method was found to be particularly effective for detecting genomic variants in a localised gene-of-interest, as opposed to the DDAT method which gives whole genome coverage.

Targeted amplification of genomic regions is a method used to generate sequencing data for specific regions of the genome. This can be a useful alternative to whole genome sequencing if the question is only whether specific genes are mutated. For example, there are known mutational hot spots in many types of cancer; taking the TET2 gene as an example, the coding regions (exons) of this gene are mutated in around 15% of patients with myeloid cancer. Rather than sequencing the whole genome (3 billion base pairs), targeted sequencing can be used for a few thousand base pairs, dramatically reducing the cost of sequencing whilst increasing the depth of information generated at the required targets. A larger number of reads covering specific areas (increased coverage), results in greater confidence in identifying true genetic variants, which may be important in driving cancer processes. Additionally, the data generated from targeted sequencing for panels of genes is now used in the clinic to help clinicians to decide on the most appropriate treatment for the patient.

### Materials and Methods

The method described for DDAT can be optimised to use for targeted DNA adapter tagging (TDAT). To demonstrate the feasibility of the method, genomic DNA extracted from the KG-1 cell line was sonicated to shear DNA to lengths that simulate good quality FFPE (1000 bp fragments on average). For the first strand synthesis, 143 primers were designed to cover exons of the TET2 gene (approximately 6013 bp in total). TET2-specific sequences of 18 bp to 22 bp were designed approximately 80-100 bp apart on both DNA strands using an online primer tiling tool. The inventors added the Illumina adapter to the 5′ end of each TET2-specific sequence (Table 5).

TABLE 5
Sequences of 1^{st} and 2^{nd} strand synthesis primers used for TDAT

| | |
|---|---|
| 1^{st} strand | 5′-CAGACGTGTGCTCTTCCGATCTN₁₈₋₂₂-3′ |
| synthesis | N₁₈₋₂₂ = TET2-specific sequence, e.g. |
| primers | TTGAGATATGCCCATCTCCT |
| 2^{nd} strand | 5′-CTACACGACGCTCTTCCGATCT, |
| synthesis | |
| primer | |

The 1^{st} strand synthesis primers containing the TET2-specific sequences and the P7 truncated Illumina adapter were mixed with 50 ng of sheared DNA extracted from KG-1 cells in 50 the mixture heated for 2 min at 95° C. and cooled at 0.1° C. per second to promote on-target annealing of the primers. The DNA/primer mixture was purified using AmpureXP beads before treatment with exonuclease I to remove excess, non-annealed 1^{st} strand synthesis primers, which helps reduce non-specific (i.e. non-TET2) binding of primers in the genome.

The 1^{st} strand synthesis of new DNA was then performed as described for DDAT, using the Klenow fragment and a slow ramp rate from 4° C. to 37° C. as described. The subsequent steps for 2^{nd} strand synthesis were performed as described for DDAT, using the 2^{nd} strand synthesis primer shown in Table 5. The final PCR amplification to create the sequencing library was 20 cycles as the region amplified is only 6013 bp.

For TDAT, the 1^{st} strand synthesis primers containing the TET2-specific sequences were attached to a truncated section of the Illumina adapter that makes up the P7 side of the adapter molecule (P7 side underlined:

5′-CAGACGTGTGCTCTTCCGATCTN₁₈₋₂₂-3′).

The 2^{nd} strand synthesis primer contains a truncated section of the P5 side of the Illumina adapter (P5 side underlined:

5′-CTACACGACGCTCTTCCGATCTNNNNNNNNN-3′),

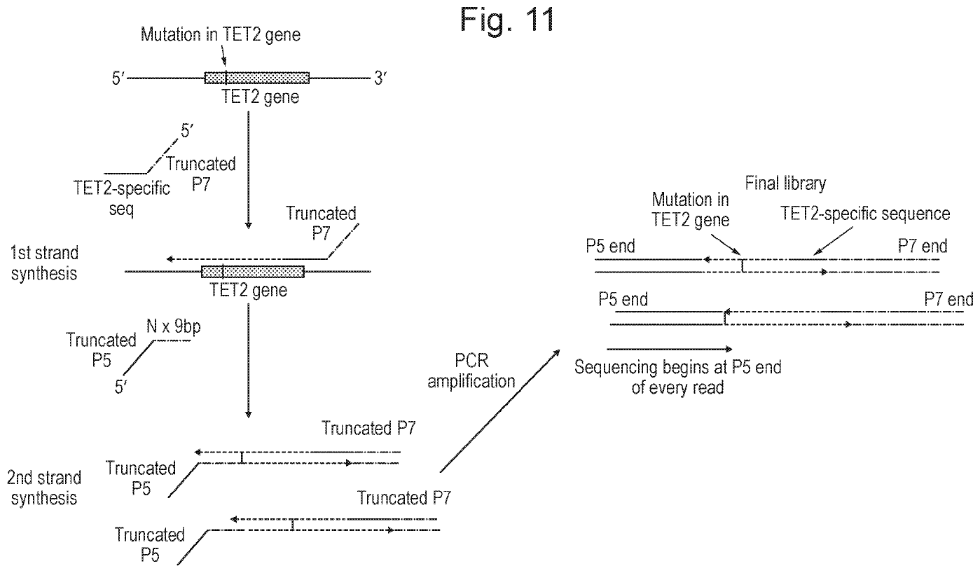

attached to the 9 random bases, therefore the 2^{nd} strand synthesis primer can anneal at a random position on the new DNA strand created during the 1^{st} strand synthesis (FIG. 11, left). When the DNA library is generated during the PCR reaction, only sequences containing both the truncated P5 and P7 will be amplified (FIG. 11, right). The sequencing of the final library on the Illumina instrument always generates data from the P5 end first, therefore the first read will always start from a random sequence of the TET2 gene, rather than containing the TET2-specific sequence (FIG. 11, right). This is an advantage for several reasons; it maintains a high level of sequence diversity during the first sequencing cycles, reducing the risk of low sequencing yield or data quality (https://emea.support.iullumina.com/bulletins/2016/07/what-is-nucleotide-diversity-and-why-is-it-important.html). It also improves the % of bases covered at the target gene and helps increase the chance of identifying a mutation, which will not always be located close to the TET2-specific sequence (FIG. 11).

### Results

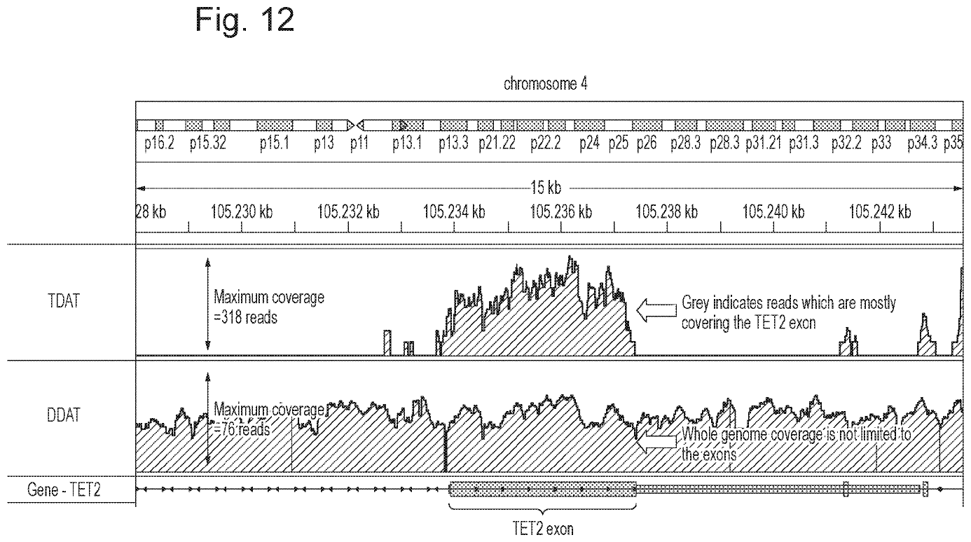

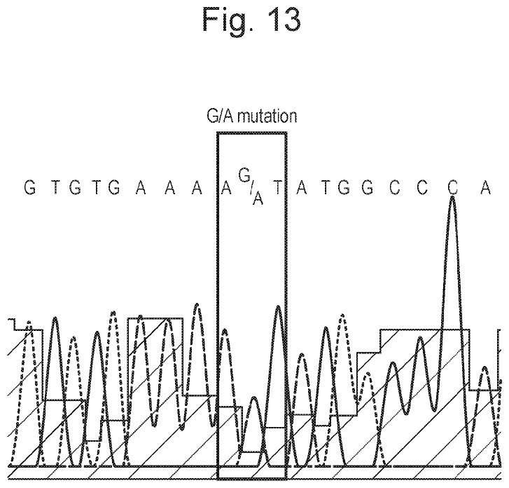

The targeted sequencing data was aligned to the human genome version hg38 using BWA (version 0.7.17.4). By visualising the data using the Integrative Genomics Viewer (IGV) it was clear that the data generated using TDAT was specific to the TET2 exons (FIG. 12, top panel), and not the whole genome, as seen from the data generated with DDAT (FIG. 12, bottom panel). The maximum coverage at TET2 exons was also greater when using TDAT (318 reads vs. 76 reads shown in FIG. 12).

TABLE 6
A summary of the sample alignment metrics for TDAT

| | |
|---|---|
| Total number of reads | 2,410,744 |
| High quality reads (MAPQ ≥20 (%)) | 88 |
| Mapped reads (%) | 65.5 |
| Unmapped reads (%) | 34.5 |
| Duplicate reads (%) | 1.9 |
| on-target reads - TET2 exons (%) | 0.3 |
| Average coverage across TET2 exons (reads/base) | 49 |
| Bases covered >8 reads (% of total) | 88.5 |
| High quality reads (MAPQ ≥20 (%)) | 88 |

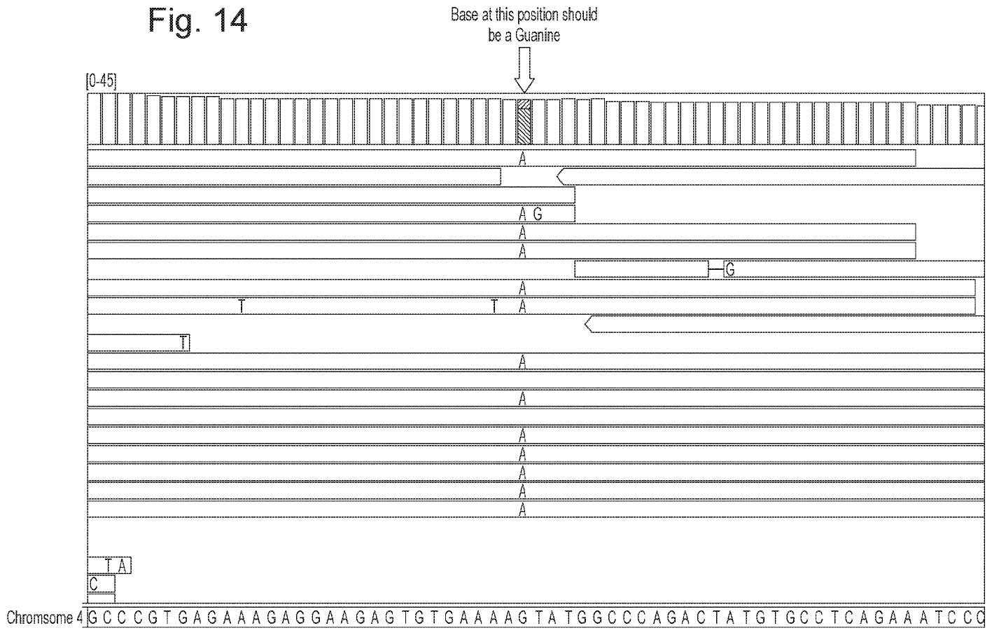

The inventors assessed the alignment metrics using QualiMap BamQC (version 2.2.2; Table 2). The analysis showed that 65.5% of reads mapped to the genome, although only 0.3% were on-target reads, mapping to TET2 exons. Typically, one would expect around 50% on-target coverage at this quantity of input DNA. Nonetheless, the average coverage across TET2 exons was 49 reads per base with 88.5% of bases covered with at least 8 reads. This is sufficient coverage to perform variant detection for mutations with a high variant allele frequency (VAF). As this was sequencing data from a cell line, we aimed to detect a known mutation in a TET2 exon, which the inventors had validated previously using sanger sequencing (FIG. 13). The inventors used Varscan (version 2.4.2) to analyse the data and confirmed a G/A mutation at chr4:105276312 (p=1.62¹⁰⁻²) (FIG. 14).

The inventors then used Varscan to analyse all the TET2 exons and identified two further single nucleotide polymorphisms (SNPs), which were not previously known in KG-1 cells. The inventors confirmed that these are known mutations that are found in humans using the Cosmic database (Table 7).

TABLE 8
Details of two SNPs in TET2 exons in KG-1 cells identified using TDAT and Varscan analysis.

| | | | Prevalence in | | | |
|---|---|---|---|---|---|---|
| | | p value calculated | general | Impact on amino | | |
| SNP ID | Mutation | from varscan analysis | population | acid | Location chr4 | COSMIC ID |
| rs3733609 | T/C | 1.7¹⁰⁻⁷ | 5.71% | Synonumous | 105269705 | COSV54402591 |
| rs6843141 | G/A | 3.9¹⁰⁻⁷ | 5.68% | Missense variant | 105234594 | COSV54412627 |

### Conclusion

In conclusion the inventors have shown that the method for DDAT can be adapted for targeted DNA adapter tagging (TDAT) to generate sequencing data for specific genes from low DNA input. It was possible to use this data to identify previously unknown mutations in the KG-1 cell line, which are verified SNPs in the human genome. Although the on-target reads to TET2 were low at 0.3% (the optimum is around 50%), this could likely be improved by more stringent primer design and using more primers in the experiment. Previously studies using related methods have used 14,000 primers when performing targeted sequencing on low DNA input, it may be that 143 primers was too few to generate 50% on-target reads when starting from a low input.

SEQUENCES

SEQ ID NO: 1
CTACACGACGCTCTTCCGATCTNNNNNNNNN
SEQ ID NO: 2
CAGACGTGTGCTCTTCCGATCTNNNNNNNNN
SEQ ID NO: 3
AATGATACGGCGACCACCGAGATCTACACTCTTTCCCTACACGACGCTC
TTCCGATCT
SEQ ID NO: 4
CAAGCAGAAGACGGCATACGAGATCGTGATGTGACTGGAGTTCAGACGT
GTGCTCTTCCGATCT
SEQ ID NO: 5
CAGACGTGTGCTCTTCCGATCTTTGAGATATGCCCATCTCCT
SEQ ID NO: 6
CTACACGACGCTCTTCCGATCNNNNNNNNN

1. A method for generating a population of double-stranded polynucleotide molecules from a sample containing at least one polynucleotide, which method does not comprise bisulfite treatment of said at least one polynucleotide, and which method comprises the steps of:
a. Denaturing said at least one polynucleotide to produce single stranded polynucleotide;
b. Incubating the single stranded polynucleotide from step a. with a first single-stranded oligonucleotide comprising a sequencing adaptor sequence and a primer sequence under conditions suitable for annealing of the first single-stranded oligonucleotide to the single stranded polynucleotide of step a., and then extending the primer with a polymerase to produce double-stranded polynucleotide;
c. Denaturing the double-stranded polynucleotide of step b. to produce single stranded polynucleotide;
d. Incubating the single stranded polynucleotide from step c. with a second single-stranded oligonucleotide comprising a sequencing adaptor sequence and a primer sequence under conditions suitable for annealing of the second single-stranded oligonucleotide to the single stranded polynucleotide of step c., and then extending the primer with a polymerase to produce a population of double-stranded polynucleotide molecules.

2. The method according to claim 1, wherein said at least one polynucleotide in the sample is RNA or DNA and/or wherein the population of double-stranded polynucleotide molecules is RNA or DNA.

3. The method according to claim 1, wherein said at least one polynucleotide is RNA, the polymerase in step b. is a reverse transcriptase, and the DNA molecules in the population generated by the method are double stranded cDNA molecules.

4. The method according to claim 1, wherein the sample contains a low quantity of DNA and/or low quality DNA, optionally wherein the sample contains less than around 1 μg, preferably less than around 200 ng, most preferably between around 2 ng to around 10 ng of DNA, and/or wherein a significant proportion of the DNA is fragmented, damaged and/or in single-stranded form.

5. The method according to claim 1, wherein the sample is of formalin-fixed and paraffin embedded (FFPE) material.

6. The method according to claim 1, wherein prior to the first denaturing step, the method comprises:
extracting at least one polynucleotide from the sample; and/or
removing damaged bases from the at least one polynucleotide with at least one base excision repair enzyme, which is optionally a DNA glycosylase, preferably selected from Single-strand selective monofunctional uracil DNA glycosylase (SMUG1) and/or Formamidopyrimidine DNA glycosylase (FPG).

7. The method according to claim 1, wherein:
Step b. further comprises purifying the single stranded polynucleotide that is annealed to the first single stranded oligonucleotide and/or the removal of any remaining single stranded oligonucleotide with an exonuclease and/or purifying the double stranded polynucleotide; and/or
Step d. further comprises purifying the single stranded polynucleotide that is annealed to the first single stranded oligonucleotide and/or the removal of any remaining single stranded oligonucleotide with an exonuclease and/or comprises purifying the double stranded polynucleotide;
wherein said purifying in either step optionally uses solid phase reversible immobilisation (SPRI) beads.

8. The method according to claim 1, which additionally comprises:
e. Amplifying the double stranded polynucleotide of step d. by polymerase chain reaction (PCR), typically for 8-12 cycles; and optionally
f. Sequencing the DNA;
wherein steps e. and f. use primers complementary to at least part of the sequencing adaptor sequences of the first and/or second single stranded oligonucleotides.

9. The method according to claim 1, wherein the extending of step b. and/or step d. is conducted by incubating the single stranded polynucleotide and the polymerase with a suitable reaction mixture at approximately 4° C., before slowly increasing the temperature up to the optimal operating temperature of the polymerase and holding at said optimal operating temperature until extension is substantially complete.

10. The method according to claim 9, wherein the optimal operating temperature of the polymerase is around 37° C. and wherein the temperature is increased to this temperature at a rate of no more than around 4° C./minute.

11. The method according to claim 10, wherein the polymerase is a Klenow DNA polymerase.

12. The method according to claim 1, wherein the primer in the first single-stranded oligonucleotide and/or the primer in the second single-stranded oligonucleotide is:
i. A random primer sequence, optionally comprising a random nonamer oligonucleotide sequence; or
ii. A primer sequence specific to a region of interest within the polynucleotide, optionally comprising a 20 mer oligonucleotide sequence

13. The method according to claim 1, wherein the sequencing adaptor sequence of the first and/or second single stranded oligonucleotide includes one or more of:
a sequence complementary to a sequencing primer;
a sequence complementary to an amplification primer;
a barcode or index sequence; and/or
a sequence to facilitate attachment to a solid surface, optionally wherein said sequence is complementary to an oligonucleotide attached to said surface.


SEQUENCE LISTING (6 sequences)

SEQ ID NO: 1 - DNA, 31 bp, Artificial Sequence
  Oligonucleotide primer
  misc_feature (23)..(31): wherein each “N” can be any nucleotide
ctacacgacgctcttccgatctnnnnnnnnn

SEQ ID NO: 2 - DNA, 31 bp, Artificial Sequence
  Oligonucleotide primer
  misc_feature (23)..(31): wherein each “N” can be any nucleotide
cagacgtgtgctcttccgatctnnnnnnnnn

SEQ ID NO: 3 - DNA, 58 bp, Artificial Sequence
  Oligonucleotide primer
aatgatacggcgaccaccgagatctacactctttccctacacgacgctcttccgatct

SEQ ID NO: 4 - DNA, 64 bp, Artificial Sequence
  Oligonucleotide primer
caagcagaagacggcatacgagatcgtgatgtgactggagttcagacgtgtgctcttccgatct

SEQ ID NO: 5 - DNA, 42 bp, Artificial Sequence
  Oligonucleotide primer
cagacgtgtgctcttccgatctttgagatatgcccatctcct

SEQ ID NO: 6 - DNA, 31 bp, Artificial Sequence; identical to SEQ ID NO: 1
  Oligonucleotide primer
  misc_feature (23)..(31): wherein each “N” can be any nucleotide